(19) United States
(12) Patent Application Publication
(10) Pub. No.: US 2006/0235495 A1
(43) Pub. Date: Oct. 19, 2006

(54) Long-acting flexible thermal compress

(76) Inventor: Cheng-Ta Tsai, Tainan (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/107,747

(22) Filed: Apr. 18, 2005

(51) Int. Cl. A61F 7/00; A61F 7/12

(52) U.S. Cl. 607/96; 607/114

(57) ABSTRACT

A long-acting flexible thermal compress includes an inner compound layer, an outer compound layer, and a thermal filling. The inner compound layer comprises a film layer laminated with a contact layer. The outer compound layer comprises a film layer laminated with a separation layer. The thermal filling is sealed in a space defined by both inner and outer compound layers welded at the peripherals of their respective film layers. The separation layer helps achieve long-acting thermal compress. The contact layer is made of a material with better touch and proper heat transfer properties to prevent burn or frostbite.

### BACKGROUND OF THE INVENTION

(a) Field of the Invention

The present invention relates to a long-acting, flexible thermal compress, and more particularly, to one has a leak-free thermal filling made integrated in a thermal compress, allows diversified applications, and prevents burn or frostbite.

(b) Description of the Prior Art

Thermal compresses generally available in the market give a comprehensive range of applications including bringing down a fever, hot compress in case of inflammation or swell, or keeping beverages or liquors cool at a proper temperature. As illustrated in FIG. 11 for a Taiwanese Utility Model Publications No. 335685 titled “Improved Structure of Hot Compress”, it comprises a soft rubber bag (A) containing a low-molecular and low-density thermal filling (A1); and a bad (A) is then sealed by welding. However, the bag (A) comprises only a single upper layer and a single lower layer to separate the filling (A1). Therefore, the heat is transferred at a very high rate. When applied on the human body, the risk of getting burnt or frostbitten. To prevent the burn or frostbite, an additional piece of fabric or towel must be used between the soft rubber bag (A) and the affected area of the skin to serve as a protection layer by preventing fast heat transfer. Meanwhile, the use of the fabric or towel is still outperformed by the heat transfer speed to fail the thermal results. Losing of the thermal effects remains a matter of few minutes. Furthermore, when used as a cold compress, the fabric, towel could get wetted into a mess.

Accordingly, as illustrated in FIG. 12, a Taiwanese Utlility Model Publication No. 443307 titled “Long-Acting Thermal Beverage Bag” comprises an enclosed packaging bag (B) containing a thermal filling (B1). Wherein, only a separation layer (B2) is provided on one side of the bag (B); and on the other side of the bag (B) is provided with a separation layer (B2), a thermal layer (B3), and an outer layer (B4) made of non-woven fabric. The single separation layer (B2) on one side of the bag (B) is attached to a container for direct heat transfer while the separation layer (B2), the thermal layer (B3), and the outer layer (B4) on the other side of the bag (B) are to slow down the loss of thermal energy to keep the beverage staying for a longer time at the temperature as desired. However, the single separation layer (B2) of the bag (B) transfers the heat at fast rate to prevent it from serving as a thermal compress for the human body.

As taught in U.S. Pat. No. 6,830,582, a therapeutic bag includes a mouth and an interior containment pocket, wherein its heat filling and the therapeutic bag are not made integrated. In another U.S. Pat. No. 6,589,272, a cold pack is an independent unit from a main body. When in use, the cold pack must be put in the main body. Another U.S. Pat. No. 6,149,617 yet, both a thermal pack and a main body are also separated from each other. In the structure of any of the prior arts quoted above, the thermal pack and the main body are made independent from each other to cause the presence of spacing between the pack and the main body; and the spacing is blamed for poor heat transfer and poor results of thermal compress.

Furthermore, as taught in U.S. Pat. No. 4,920,964, a multi-layer structure composed of a nylon layer and a PE layer for a thermal compress; however, direct use of nylon is more likely to cause burn or frostbite due to the absence of thermal retaining property of nylon, resulting in fast heat dispense to allow only short acting time.

### SUMMARY OF THE INVENTION

A preferred embodiment of the present invention related to a long-acting thermal compress that is applicable to the occasion wherein contact of human skin is needed includes an inner compound layer, an outer compound layer and a thermal filling. The inner compound layer comprises a film layer laminated with a contact layer made of a material providing better touch and proper heat transfer property. The outer compound layer comprises a film layer laminated with a separation layer made of a material providing low heat transfer property. Both the inner and the outer compound layers are welded to each other at the peripherals of their respective film layers to define a sealed space containing the thermal filling. In this preferred embodiment, the film layer may be made a single layer film or a multi-layer film. The multi-layer film is in the form of laminated film or co-ex casting film.

Another preferred embodiment of the present invention is a long-acting thermal compress that is applicable to the occasion wherein contact of human skin is not needed includes an inner compound layer, an outer compound layer and a thermal filling. Wherein, the inner compound layer is a leak-proof film layer. The outer compound layer comprises a film layer laminated with a separation layer made of a material providing low heat transfer property. The inner compound layer and the outer compound layer are welded to each other at the peripherals of their respective film layers to define a sealed space containing the thermal filling. Accordingly, the thermal filling is integrated with the thermal compress into one piece. In this preferred embodiment, the film layer can be a multi-layer film in the form of laminated film or co-ex casting film.

The primary purpose of the present invention is to provide a long-acting, flexible thermal compress with its thermal filling made integrated with the compress to permit convenient use.

Another purpose of the present invention is to provide a long-acting, flexible thermal compress that provides good touch, extends thermal time, and prevents burn or frostbite in use.

Another purpose yet of the present invention is to provide a long-acting, flexible thermal compress that gives good flexible characteristic to perfectly compromise the affected part of a human body.

The present invention when adapted with an additional member or made in various forms allows to be applied in position on many parts of the human body for alleviating fever, inflammation, pains, and thermal purposes without falling off or getting loosened up while maintaining nice and tidy appearance.

In addition to being applied on the human body, the present invention by taking advantage of its flexibility can be made in tubular shape to wrap up canned beverage for thermal purpose.

Furthermore, the outer compound layer may be printed with or adhered with a layer of pattern.

the present invention in general provides the following advantages:

1. Integrated in one piece. With the built-in thermal filling to eliminate any spacing between the inner and the outer compound layers, the present invention allows consistent heat transfer and easy use.

2. Long-acting. With the presence of the separation layer, the present invention permits extended time of thermal accumulation for improved thermal results.

3. Proper heat transfer. The present invention by allowing direct contact between the contact layer with better touch and the skin provides excellent thermal results neither causing burn or frostbite nor the use of any towel or fabric as a pad.

4. Fast. With the aid of a magic tape, the present invention can be fastened to many parts of the human body without loosening up or falling off.

5. Comprehensive use. While alleviating fever, congestion, inflammation and keeping warm, the present invention can be used as a seat cushion and for keeping the beverage at the thermal temperature as desired.

6. Attractive appearance. The present invention provides attractive appearance by printing on or sticking to the outer compound layer an elegant pattern layer.

7. Flexibility. The present invention provides good flexibility to fully compromise the human body for giving the excellent thermal results.

### BRIEF DESCRIPTION OF THE DRAWINGS

FIG. 1 is an exploded view of a preferred embodiment of the present invention.

FIG. 2 is a sectional view of an assembly of the preferred embodiment of the present invention.

FIG. 3 is a schematic view of the preferred embodiment of the present invention adapted with a magic tape.

FIG. 4 is a schematic view showing that the preferred embodiment of the present invention is used on one's head.

FIG. 5 is a schematic view showing that the preferred embodiment of the present invention is used on one's extremities.

FIG. 6 is a schematic view showing that the preferred embodiment of the present invention is used on insoles.

FIG. 7 is a schematic view showing that the preferred embodiment of the present invention is used on a seat cushion.

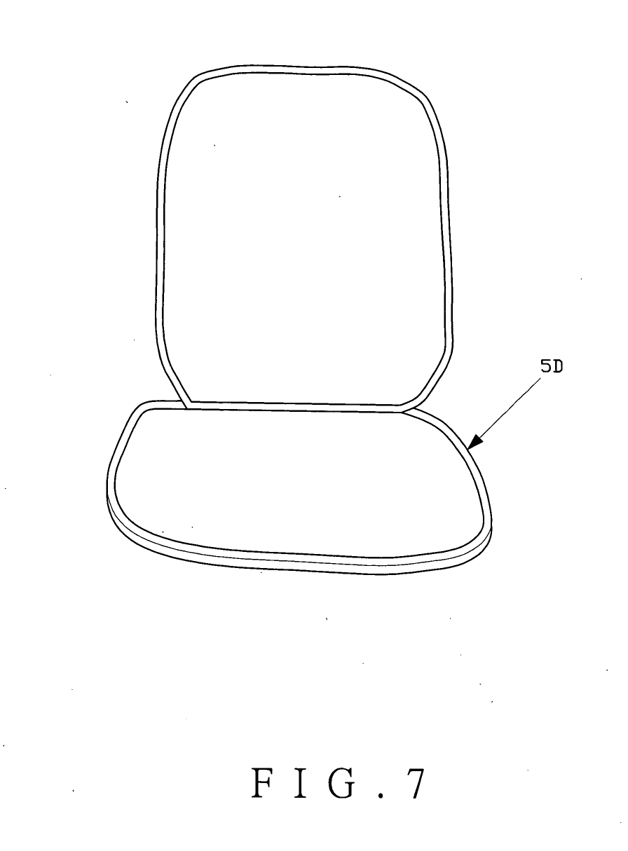

FIG. 8 is a perspective view of another preferred embodiment of the present invention.

FIG. 9 is a schematic view showing that the present invention is used on beverages.

FIG. 10 is a schematic view showing that the present invention is applied with an additional layer of pattern.

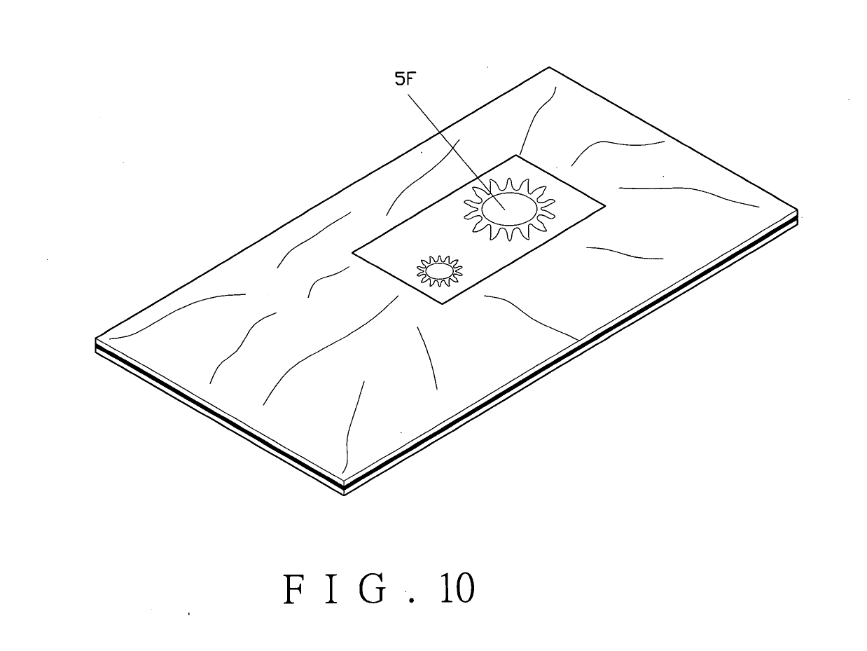

FIG. 11 is a sectional view of a prior art.

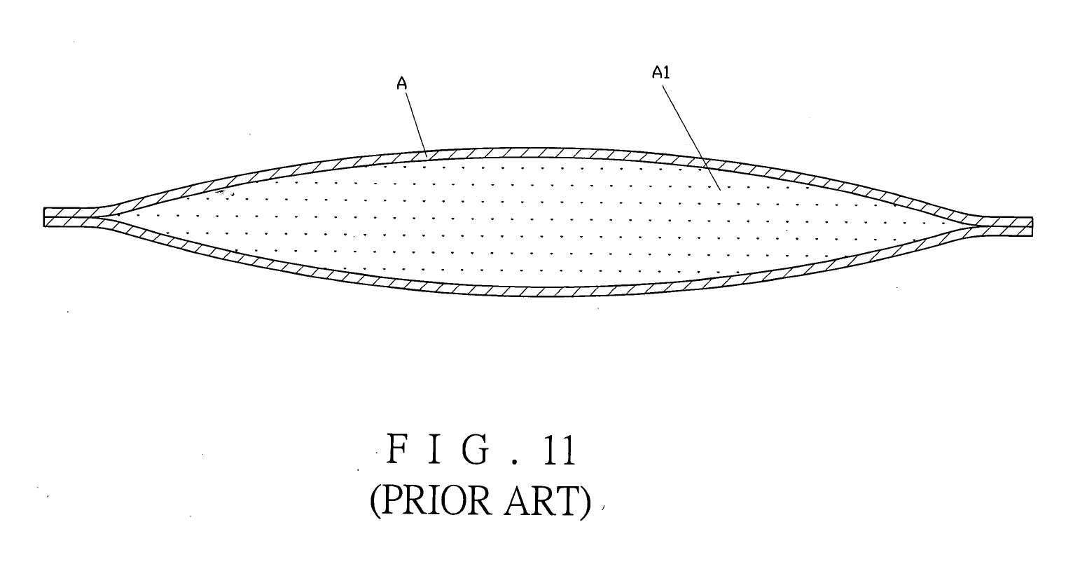

FIG. 12 is a sectional view of another prior art.

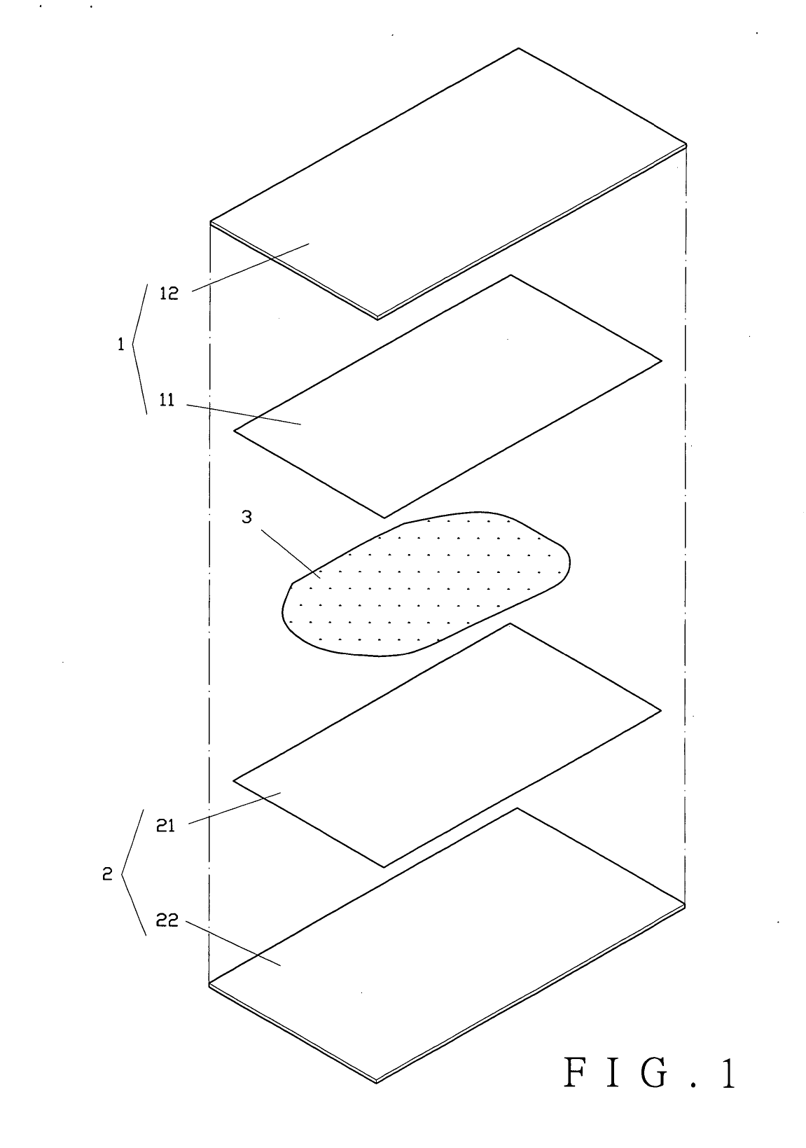

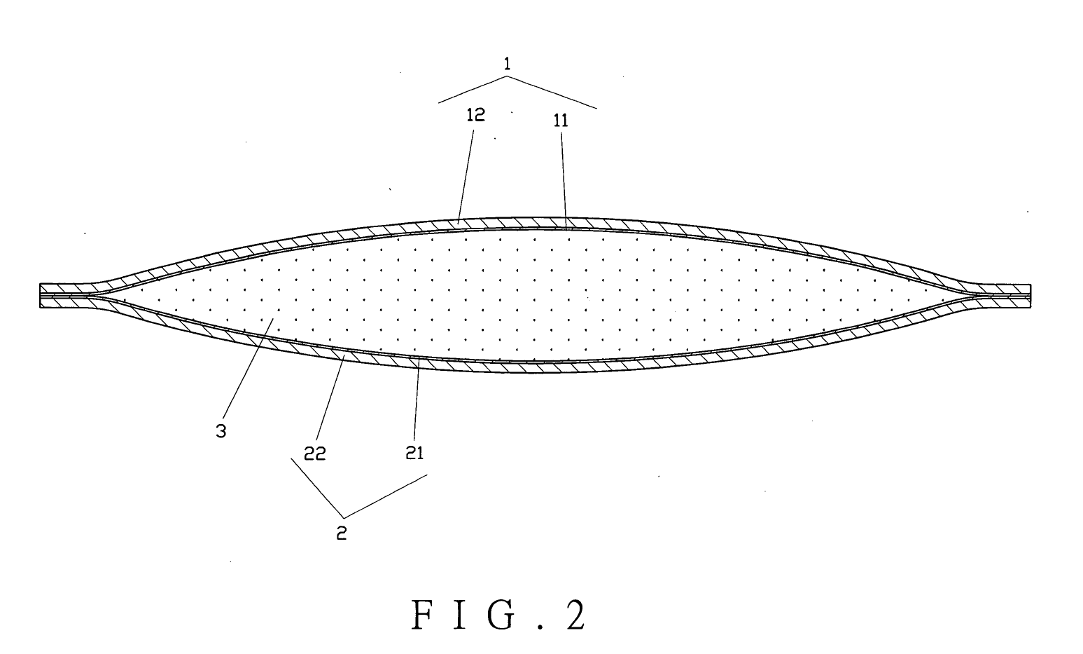

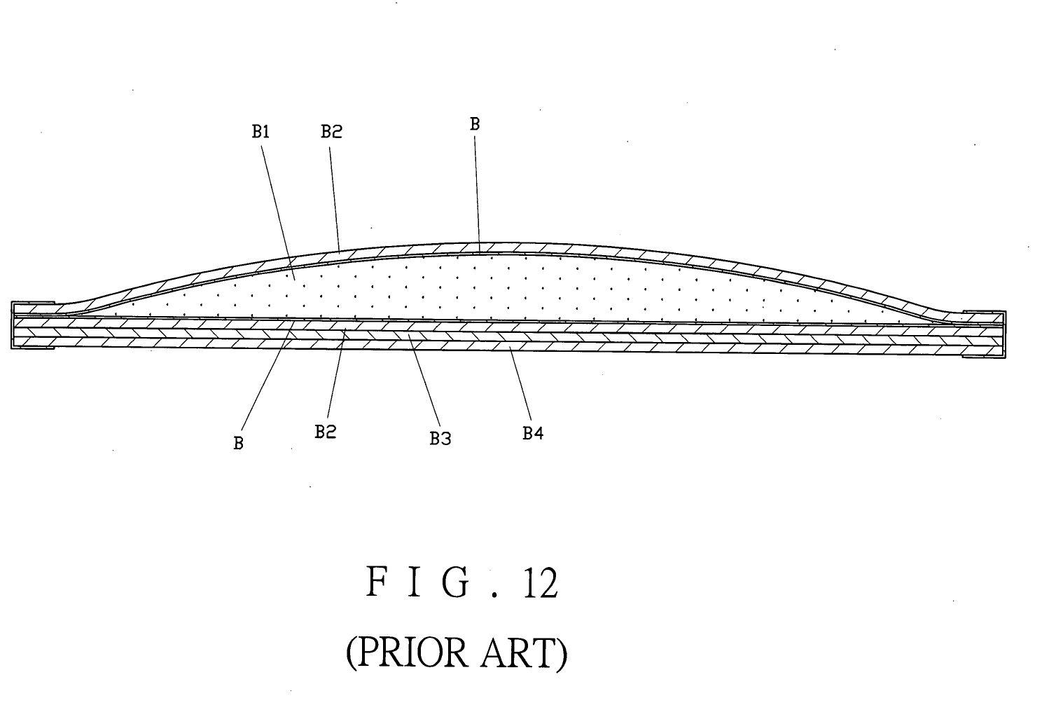

Referring to FIGS. 1 and 2, a first preferred embodiment of the present invention comprises an inner compound layer (1), an outer compound layer (2) and a thermal filling (3).

The inner compound layer (1) is flexible and comprises a film layer (11) laminated with a contact layer (12). The film layer (11) may be a single layer film or multi-layer film. The film layer (11) and the contact layer (12) are laminated to each other by means of adhesive, solvent, solvent-free or coating process. The film layer (11) preferred in a thickness of 40μm ˜500μm is a leak-proof single layer film or a multi-layer film providing freeze and thermal resisting properties without breaking up between a work temperature of −20° C.˜100° C. . The contact layer (12) is preferred made of a material with better touch, e.g., knitted or woven fabric to prevent from burn or frostbite upon contacting human skin while serving as a protection layer for the film layer (11).

The outer compound layer (2) to be sealed with the inner compound layer (1) is flexible and comprises a film layer (21) laminated with a separation layer (22). The film layer (21) may be a single layer film or multi-layer film. The film layer (21) and the separation layer (22) are laminated to each other by means of solvent-based, solvent-free based lamination or coating process. The film layer (21) is in a thickness of 0.35 mm˜8 mm and a leak-proof single layer film or a multi-layer film providing freeze and thermal resisting properties without breaking up between a work temperature of −20° C.˜100° C. . The separation layer (22) is made of thermal material, e.g., natural or synthetic rubber, or EVA foam, in a thickness of 0.35 mm˜8 mm to provide extended thermal time, prevent fast lose of thermal energy, while preventing leakage and protecting the film layer (21) from being pierced through.

The thermal filling (3) is sealed between the inner compound layer (1) and the outer compound layer (2).

The film layer (11) may be PE, (Polyethylene), PP (Polypropylene), LLDPE (Linear Low Density Polyethylene), or CPP (Crosslink Polypropylene) single layer film.

The film layer (11) may be also a Nylon/PE, Nylon/CPP, Nylon/LLDPE multi-layer film, and the multi-layer film may be a laminated film or a co-ex casting film.

The film layer (11) may be also a single layer of elastomer film or multi-layer elastomer film, such as TPV (Thermoplastic Vulcanizate), TPU (Thermo Poly Urethane), or TPE (Thermo Plastic Elastomer).

The separation layer (22) may be made of less elastic heat insulation material, e.g., EPE (Polyethylene Foam), IXPE (Irradiated cross-linked Polythylene), XPE (Cross-linked Polyethylene), or EVA (Ethylene-vinyl Acetate Copolymer) foam; or made of elastic heat insulation material, e.g., natural rubber, synthetic rubber, neoprene, TPE (Thermo Plastic Elastomer), SBC (Styreneic Block Copolymers), SEBS (Styrene Ethylene Butylene Styrene), or Copolymer Compound.

Both the inner compound (1) and the outer compound (2) are separately produced and cut into a given shape before being overlapped and welded to each other on their peripherals by the hot welding meld, ultra sonic welding, or high frequency welding process to define a sealed, leak-proof space between the inner compound layer (1) and the outer compound layer (2). In the preferred embodiment, the hot plate welding method is used to seal the respective peripherals of the inner compound layer (1) and the outer compound layer (2). The hot plate welding method involves the use of heating tube or heating play in conjunction with a temperature control box to reach the pre-set welding temperature. A mouth must be reserved in the course of the welding for pouring into the thermal filling (3). The welding temperature used for the sealing is about 30° C. higher than the melting points of the film layers (11, 21) of the inner and the outer compound layers (1, 2). A proper pressure is applied to hold from top the inner compound layer (1) for the heat generated from the heating plate to create heat transfer through the contact layer (12) of the inner compound layer (1) for both the film layers (11, 12) of the inner and the outer compound layers (1, 2) to be welded and sealed to each other. The mouth is reserved each to the inner and the outer compound layers (1, 2) for pouring the thermal filing (3) and the mouth is then sealed by using the same welding method to complete the production of a thermal compress of the present invention

When the preferred embodiment is used on the human body, the compress is heated or cooled down (put in a micro-over or a freezer). The contact layer (12) of the inner compound layer (1) is to directly contact the skin. Whereas both the inner and outer compound layers provide excellent flexibility, the compress is applied on the human body by fully compromising the skin without creating any spacing. The separation function offered by the contact layer (12) keeps the skin from any burn or frostbite, and the separation layer (22) of the outer compound layer (2) provides the extended thermal result.

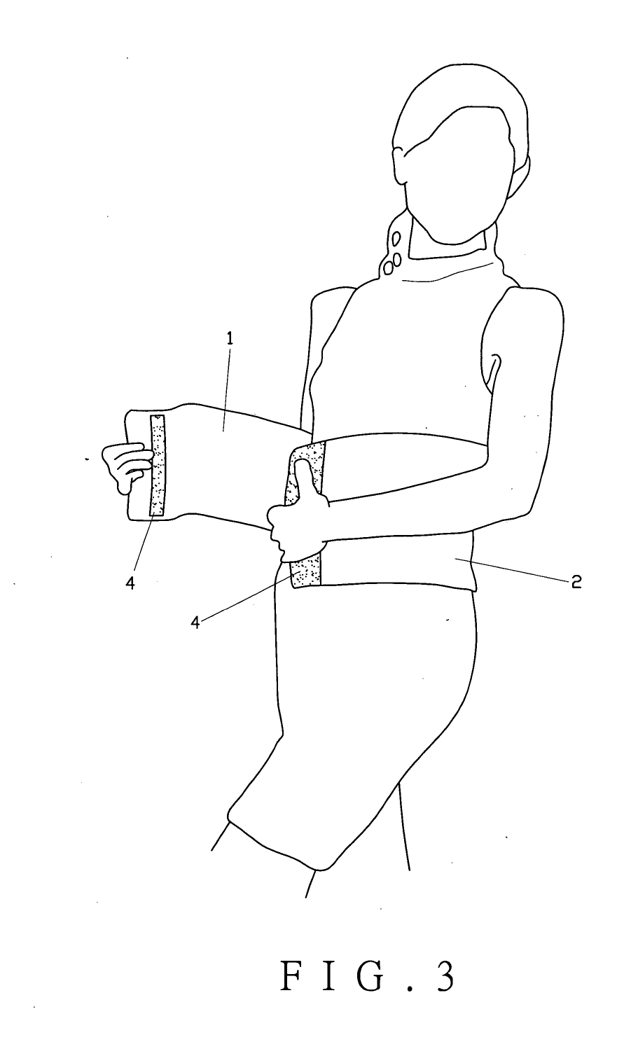

To secure the present invention to the human body, an additional attachment may be used. A magic tape (4) is used, as illustrated in FIG. 3, to firmly secure the compress of the present invention to the human body without moving around or falling off for easy application.

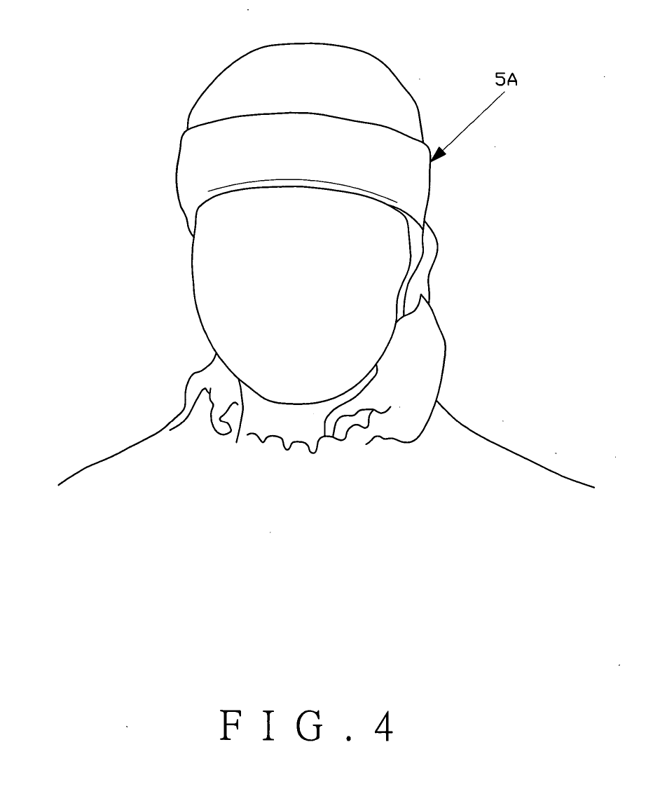

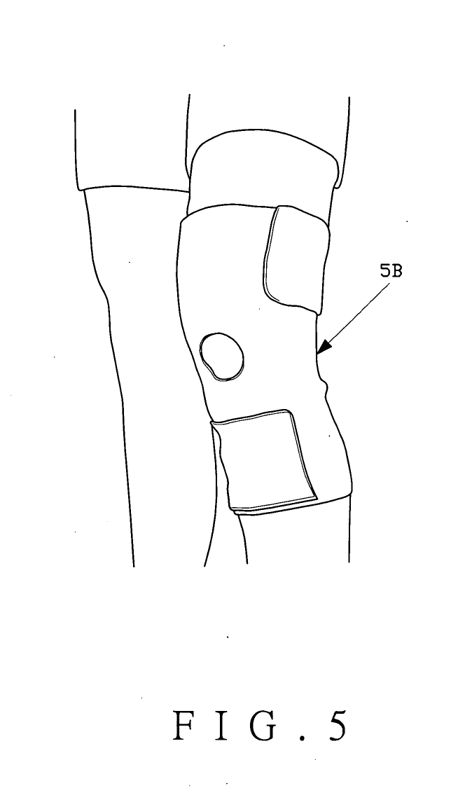

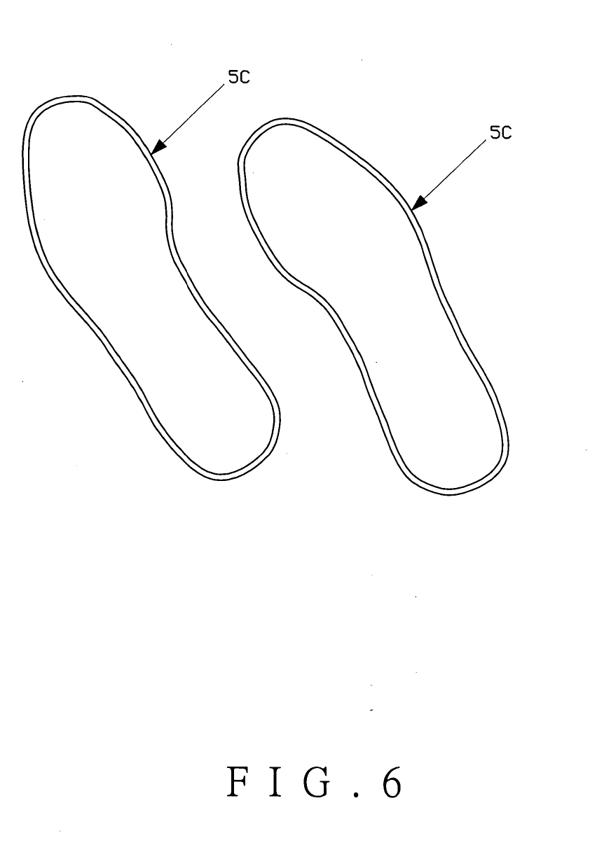

As illustrated in FIG. 4, the present invention is made into a headband (5A) for the application on the forehead to alleviate fever; in a bandage (5B) to dress muscle of the extremities that is affected with inflammation or pain as illustrated in FIG. 5; in a shape of an insole (5C) to protect the foot as illustrated in FIG. 6; and in a seat cushion (5D) for seating comfort.

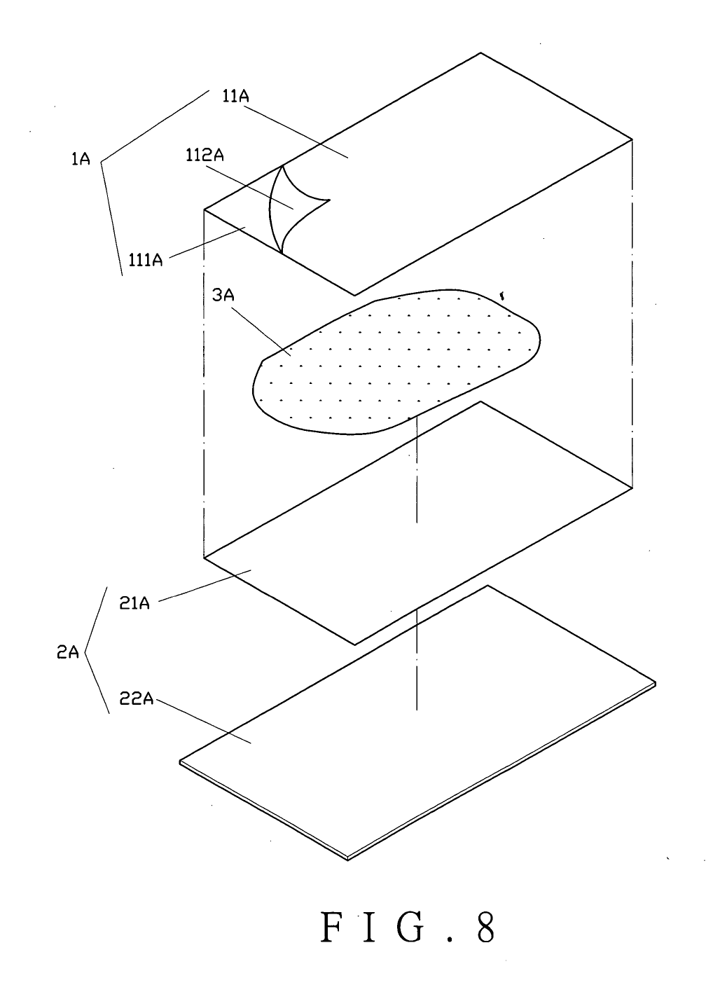

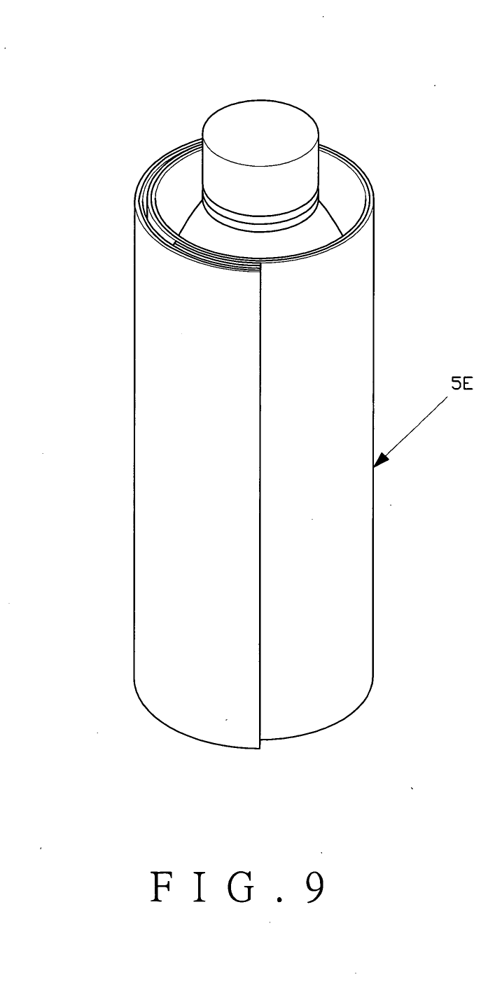

When the present invention is applied in to occasion other than contacting skin as illustrated in FIG. 8, considerations of factors of good touch and heat transfer for the inner compound layer may not be essential. Therefore, an inner compound layer comprises only a film layer (11A) in the form of a leak-proof multi-layer film. The film layer (11A) contains one or a plurality of inner layer (111A) appropriately for the welding process, and an outer layer (112A) provided with proper tensile strength and tear strength (e.g., the PE film layer allowing the welding process and Nylon film layer provided with proper tensile strength and tear strength). An outer compound layer (2A) comprises a film layer (21A) and a separation layer (22A). When molded, the inner layer (111A) and the film layer (21A) of the outer compound layer (2A) are welded to each other at their respective peripherals to define a sealed space for accommodating a thermal filling (3A). Whereas the outer layer (112A) provides proper tensile strength and tear strength, the compress as a while maintains its strength and extension as expected. As illustrated in FIG. 9, the present invention for giving excellent flexibility can be packed in a roll (5E) for wrapping up canned beverage to keep it at the hot or cold temperature as desired. Alternatively, the outer compound layer may be directly printed on a pattern or an additional layer of pattern (5F) to improve the appearance of the present invention.

I claim,

1. A long-acting, flexible thermal compress comprising:
an inner compound layer having a leak-proof film layer laminated with a contact layer, said contact layer being made of a material with better touch and proper heat transfer properties;
an outer compound layer having a leak-proof film layer laminated with a separation layer, said separation layer being made of a material with lower heat transfer property; and
a thermal filling sealed in a space defined by having said inner and outer compound layers welded at the peripherals of said respective film layers.

2. The long-acting, flexible thermal compress of Claim 1, wherein each of said film layers of said inner and outer compound layers is a single layer film.

3. The long-acting, flexible thermal compress of Claim 1, wherein each of said film layers of said inner and outer compound layers is a multi-layer film.

4. The long-acting, flexible thermal compress of Claim 1, wherein each of said film layers of said inner and outer compound layers is in a thickness of 40 μm˜500 μm.

5. The long-acting, flexible thermal compress of Claim 1, wherein each of said film layers of said inner and outer compound layers is in a thickness of 0.35 mm˜8 mm.

6. A long-acting, flexible thermal compress comprising an inner compound layer, an outer compound layer, and a thermal filling, said inner compound layer being a film layer, said film layer being is a multi-layer film; said outer compound layer having a film layer laminated with a separation layer made of a material with lower heat transfer property; and said thermal filling sealed in a space defined by having said inner and outer compound layers welded at the peripherals of said respective film layers.
